(19) United States
(12) Patent Application Publication
(10) Pub. No.: US 2013/0150965 A1
(43) Pub. Date: Jun. 13, 2013

(54) FUSION IMPLANT

(71) Applicants: Alan G. Taylor, Memphis, TN (US); Rebecca Hawkins Wahl, Escondido, CA (US); Bruce R. Lawrence, Oceanside, CA (US)

(72) Inventors: Alan G. Taylor, Memphis, TN (US); Rebecca Hawkins Wahl, Escondido, CA (US); Bruce R. Lawrence, Oceanside, CA (US)

(21) Appl. No.: 13/409,333

(22) Filed: Mar. 1, 2012

Related U.S. Application Data
(60) Provisional application No. 61/569,421, filed on Dec. 12, 2011.
(60) Provisional application No. 61/599,335, filed on Feb. 15, 2012.

(51) Int. Cl. A61F 2/28

(52) U.S. Cl. 623/16.11

(57) ABSTRACT

An implant (412) that facilitates the fusion of a first bone part (14A) with a second bone part (16A) includes an implant body (444) that extends between the first bone part (14A) and the second bone part (16A). The implant body (444) includes a first section (422) that fits within a first receiving aperture (20) in the first bone part (14A), and the implant body (444) includes a second section (426) that fits within a second receiving aperture (24) in the second bone part (16A). At least one of the first section (422) and the second section (426) can be made of bone. Additionally, at least one of the first section (422) and the second section (426) can be formed to have a non-circular cross-sectional shape to inhibit relative motion between the bone parts (14A, 16A) and the implant body (444).

### RELATED APPLICATIONS

The application claims priority on U.S. Provisional Application Ser. No. 61/569,421 filed on Dec. 12, 2011, entitled “Fusion Implant”; and on U.S. Provisional Application Ser. No. 61/599,335 filed on Feb. 15, 2012, entitled “Fusion Implant.” As far as is permitted, the contents of U.S. Provisional Application Ser. No. 61/569,421; and U.S. Provisional Application Ser. No. 61/599,335 are incorporated herein by reference.

### BACKGROUND

It is well known that some people have problems with one or more joints in their body, including in their feet and/or hands, and/or with the healing of broken bones. For example, many people suffer from potentially painful conditions with their toes, such as claw toe, mallet toe, hammer toe, or curly toe. Several procedures have been developed to treat these and other conditions and/or to treat and encourage the proper healing of broken bones. However, existing treatments are not entirely satisfactory.

### SUMMARY

The present invention is directed to an implant that facilitates the fusion of a first bone part with a second bone part. The first bone part includes a first receiving aperture and the second bone part includes a second receiving aperture. In certain embodiments, the implant comprises an implant body that extends between the first bone part and the second bone part. Additionally, the implant body includes a first section that fits within the first receiving aperture in the first bone part. Further, the implant body includes a second section that fits within the second receiving aperture in the second bone part. In some embodiments, at least one of the first section and the second section is made of bone. Additionally, in such embodiments, at least one of the first section and the second section is formed to have a non-circular cross-sectional shape to inhibit relative motion between the bone parts and the implant body.

In one embodiment, at least one of the first section and the second section has a generally rectangular cross-sectional shape. Additionally and/or alternatively, in one embodiment, at least one of the first section and the second section has a generally octagonal cross-sectional shape.

Additionally, in certain embodiments, each of the first section and the second section are made of bone. In one embodiment, at least one of the first section and the second section is made of cortical bone. Further, in one embodiment, at least one of the first section and the second section is made of bone that is partially demineralized.

Further, in one embodiment, each of the first section and the second section are formed to have a non-circular cross-sectional shape to inhibit relative motion between the bone parts and the implant body. Alternatively, in one embodiment, one of the first section and the second section is threaded and includes a substantially circular cross-section that is threaded into its corresponding receiving aperture.

In some embodiments, the implant body further includes a plurality of ridges. In such embodiments, at least one of the ridges extends substantially transverse to a longitudinal axis of the implant body.

In one embodiment, the implant body is generally straight beam shaped. Alternatively, in one embodiment, the second section is angled relative to the first section.

Additionally, in certain embodiments, the implant body can include an orientation indicator that indicates that the first section of the implant body is to be inserted into the first bone part. Moreover, in some embodiments, the implant body can further include a depth indicator that indicates when the first section is properly inserted into the first bone part. In one such embodiment, the depth indicator includes a joint line feature to indicate an insertion depth of the first section into the first bone part.

Further, the present invention is directed to a method for fusing a first bone part with a second bone part. In certain embodiments, the method comprises the steps of (i) extending an implant body between the first bone part and the second bone part, the implant body including a first section and a second section; (ii) fitting the first section within a first receiving aperture in the first bone part; and (iii) fitting the second section within a second receiving aperture in the second bone part, wherein at least one of the first section and the second section is made of bone, and wherein at least one of the first section and the second section is formed to have a non-circular cross-sectional shape to inhibit relative motion between the bone parts and the implant body.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a top view of a portion of a foot with four fusion implants having features of the present invention implanted therein;

FIG. 2 is a top view of a portion of a hand with four fusion implants having features of the present invention implanted therein;

FIG. 3 is a top view of a portion of a hand and arm with a fusion implant having features of the present invention implanted therein;

FIG. 4A is a side view of an embodiment of a fusion implant having features of the present invention;

FIG. 4B is a bottom view of the fusion implant illustrated in FIG. 4A;

FIG. 4C is an end view of the fusion implant illustrated in FIG. 4A;

FIG. 5A is a side view of another embodiment of a fusion implant having features of the present invention;

FIG. 5B is an end view of the fusion implant illustrated in FIG. 5A;

FIG. 6A is a side view of still another embodiment of a fusion implant having features of the present invention;

FIG. 6B is an end view of the fusion implant illustrated in FIG. 6A;

FIG. 7A is a side view of yet another embodiment of a fusion implant having features of the present invention; and

FIG. 7B is a perspective view of the fusion implant illustrated in FIG. 7A.

### DESCRIPTION

The present invention is directed to a fusion implant that can be used to treat and fuse two bone parts. As non-exclusive examples, the fusion implant can be used to assist in the fusion of articular joints in the forearm, wrist, hand (including fingers), lower leg, foot (including toes), and/or ankle of a human or animal. In these examples, one side of the joint being fused can be considered a first bone part, and the other side of the joint can be considered a second bone part. Additionally and/or alternatively, the fusion implant can be used in the repair of fractures of various bones in the body of a human or animal including the clavicle, humerus, ulna, radius, tibia or fibula. In these examples, one side of the fractured bone can be considered the first bone part, and the other side of the fractured bone can be considered the second bone part.

As an overview, in certain embodiments, the fusion implant can include an implant body that is made of bone. For example, as provided herein, the implant body can be made from cortical bone. Moreover, the implant body can be made of bone that is partially demineralized. In one specific example, the implant body can be a partially demineralized human cortical bone allograft. Additionally, in certain embodiments, the implant body can be formed to have a non-circular cross-sectional shape to inhibit relative movement/rotation between the bone parts and the implant body. Further, the implant body can include a plurality of ridges that inhibit the implant from being pulled out of the bone, wherein one or more of the ridges extend substantially transverse to a longitudinal axis of the implant body.

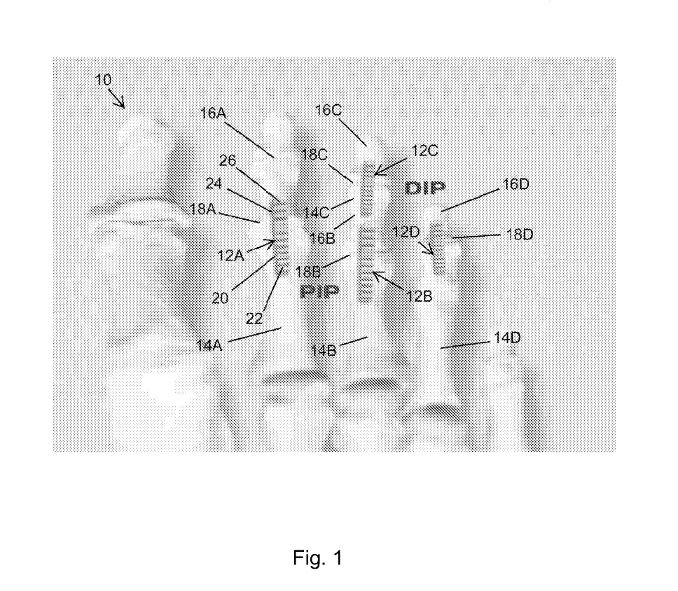

FIG. 1 is a top view of a portion of a body part 10, e.g., a portion of a right foot in this example, with four fusion implants, i.e. a first fusion implant 12A, a second fusion implant 12B, a third fusion implant 12C and a fourth fusion implant 12D (also referred to herein as an “implant” or “implants”) having features of the present invention implanted therein. More particularly, FIG. 1 illustrates (i) the first implant 12A that facilitates the fusion of a first bone part 14A (a metatarsal) and a second bone part 16A (a proximal phalanx) of a first joint 18A (proximal interphalangeal joint “PIP” of the second toe); (ii) the second implant 12B that facilitates the fusion of a first bone part 14B and a second bone part 16B of a second joint 18B (proximal interphalangeal joint of the third toe); (iii) the third implant 12C that facilitates the fusion of a first bone part 14C (a proximal phalanx) and a second bone part 16C (a distal phalanx) of a third joint 18C (distal interphalangeal joint “DIP” of the third toe); and (iv) the fourth implant 12D that facilitates the fusion of a first bone part 14D and a second bone part 16D of a fourth joint 18D (distal interphalangeal joint of the fourth toe).

In this embodiment, prior to the insertion of the implants 12A, 12B, 12C, 12D for each interphalangeal joint 18A, 18B, 18C, 18D, respectively, the proper amount of bone is removed and a first (or proximal) receiving aperture 20 is created in each of the first bone parts 14A, 14B, 14C, 14D that is sized and shaped to receive a first (or proximal) section 22 of the respective implant 12A, 12B, 12C, 12D. Somewhat similarly, the proper amount of bone is removed and a second (or distal) receiving aperture 24 is created in each of the second bone parts 16A, 16B, 16C, 16D that is sized and shaped to receive a second (or distal) section 26 of the respective implant 12A, 12B, 12C, 12D. For example, each receiving aperture 20, 24 can have a cross-section that is substantially circle-shaped, triangle-shaped, square-shaped, rectangle-shaped, hexagon-shaped, octagon-shaped, or some other shape. With this design, (i) the first implant 12A is inserted and fitted within the receiving apertures 20, 24 of the bone parts 14A, 16A, respectively, and extends between the bone parts 14A, 16A; (ii) the second implant 12B is inserted and fitted within the receiving apertures 20, 24 of the bone parts 14B, 16B, respectively, and extends between the bone parts 14B, 16B; (iii) the third implant 12C is inserted and fitted within the receiving apertures 20, 24 of the bone parts 14C, 16C, respectively, and extends between the bone parts 14C, 16C; and (iv) the fourth implant 12D is inserted and fitted within the receiving apertures 20, 24 of the bone parts 14D, 16D, respectively, and extends between the bone parts 14D, 16D.

In one embodiment, the articular joint surface of the bone parts 14A-14D, 16A-16D will be prepped by removing the damaged cartilage and then creating a hole through both opposing joint surfaces (proximal and distal) of the bone parts 14A-14D, 16A-16D. The design of the implant 12A-12D is such that the machined surface of the implant 12A-12D prevents the two opposing bone parts 14A-14D, 16A-16D from moving in translation, distraction, or rotation relative to each other. The surface shape of the implant 12A-12D also optimizes the surface area of the implant 12A-12D interfacing with the prepared bone parts 14A-14D, 16A-16D receiving the implant 12A-12D.

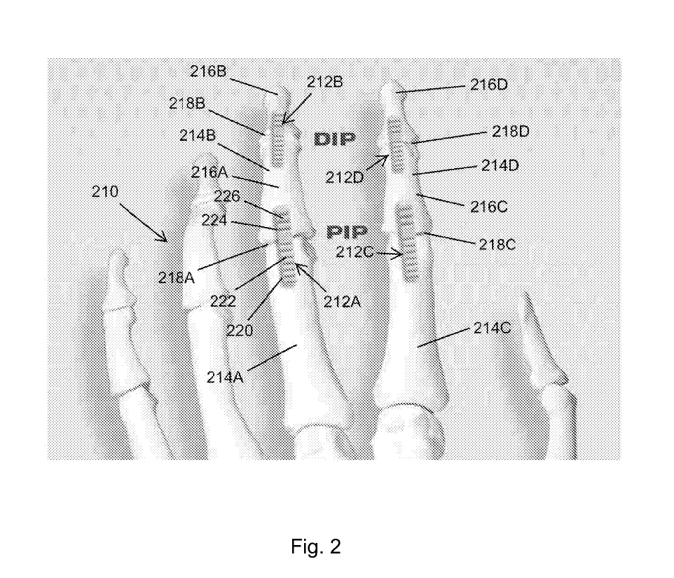

FIG. 2 is a top view of a portion of a body part 210, e.g., a portion of a left hand in this example, with four fusion implants, i.e. a first fusion implant 212A, a second fusion implant 212B, a third fusion implant 212C and a fourth fusion implant 212D (also referred to herein as an “implant” or “implants”) having features of the present invention implanted therein. More particularly, FIG. 2 illustrates (i) the first implant 212A that facilitates the fusion of a first bone part 214A and a second bone part 216A of a first joint 218A (proximal interphalangeal joint of the middle finger); (ii) the second implant 212B that facilitates the fusion of a first bone part 214B and a second bone part 216B of a second joint 218B (distal interphalangeal joint of the middle finger); (iii) the third implant 212C that facilitates the fusion of a first bone part 214C and a second bone part 216C of a third joint 218C (proximal interphalangeal joint of the index finger); and (iv) the fourth implant 212D that facilitates the fusion of a first bone part 214D and a second bone part 216D of a fourth joint 218D (distal interphalangeal joint of the index finger).

Similar to the embodiment illustrated in FIG. 1, in this embodiment, prior to the insertion of the implants 212A, 212B, 212C, 212D for each interphalangeal joint 218A, 218B, 218C, 218D, respectively, the proper amount of bone is removed and a first (or proximal) receiving aperture 220 is created in each of the first bone parts 214A, 214B, 214C, 214D that is sized and shaped to receive a first (or proximal) section 222 of the respective implant 212A, 212B, 212C, 212D. Somewhat similarly, the proper amount of bone is removed and a second (or distal) receiving aperture 224 is created in each of the second bone parts 216A, 216B, 216C, 216D that is sized and shaped to receive a second (or distal) section 226 of the respective implant 212A, 212B, 212C, 212D. For example, each receiving aperture 220, 224 can have a cross-section that is substantially circle-shaped, triangle-shaped, square-shaped, rectangle-shaped, hexagon-shaped, octagon-shaped, or some other shape. With this design, (i) the first implant 212A is inserted and fitted within the receiving apertures 220, 224 of the bone parts 214A, 216A, respectively, and extends between the bone parts 214A, 216A; (ii) the second implant 212B is inserted and fitted within the receiving apertures 220, 224 of the bone parts 214B, 216B, respectively, and extends between the bone parts 214B, 216B; (iii) the third implant 212C is inserted and fitted within the receiving apertures 220, 224 of the bone parts 214C, 216C, respectively, and extends between the bone parts 214C, 216C; and (iv) the fourth implant 212D is inserted and fitted within the receiving apertures 220, 224 of the bone parts 214D, 216D, respectively, and extends between the bone parts 214D, 216D.

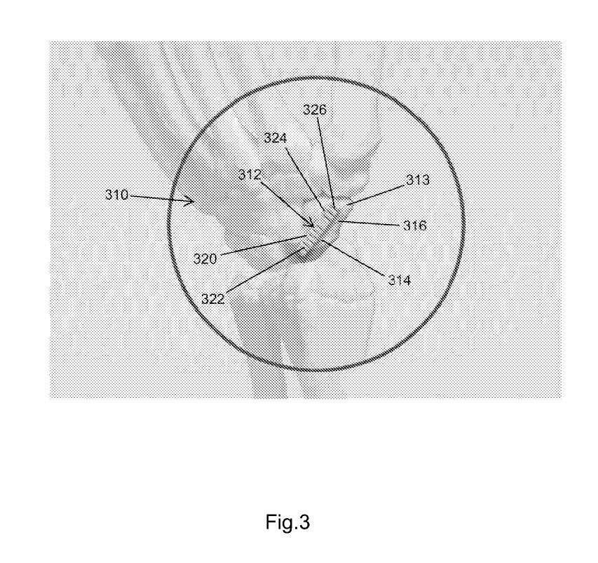

FIG. 3 is simplified illustration of a portion of a body part 310 (e.g., a portion of a hand and an arm in this example) with an implant 312 that facilitates the fusion of a break in a bone 313, i.e. the scaphoid. Alternatively, the implant 312 can be utilized to facilitate the fusion of a break in another bone, such as the clavicle, humerus, ulna, radius, tibia, fibula, or some other bone.

As illustrated in FIG. 3, a portion of the bone 313 on either side of the break can be labeled as a first bone part 314 and a second bone part 316. In particular, the fracture in the bone 313 may be repaired by inserting the implant 312 at the fracture site and fixing the first bone part 314 and the second bone part 316 together with the use of the implant 312.

In this embodiment, prior to insertion of the implant 312 to facilitate the fusion of the broken bone 313, the proper amount of the first bone part 314 is removed and a first receiving aperture 320 is created in the first bone part 314 that is sized and shaped to receive a first section 322 of the implant 312. Somewhat similarly, the proper amount of the second bone part 316 is removed and a second receiving aperture 324 is created in the second bone part 316 that is sized and shaped to receive a second section 326 of the implant 312. For example, each receiving aperture 320, 324 can have a cross-section that is substantially circle-shaped, triangle-shaped, square-shaped, rectangle-shaped, hexagon-shaped, octagon-shaped, or some other shape. With this design, the implant 312 is inserted and fitted within the receiving apertures 320, 324 of the bone parts 314, 316, respectively, and extends between the bone parts 314, 316.

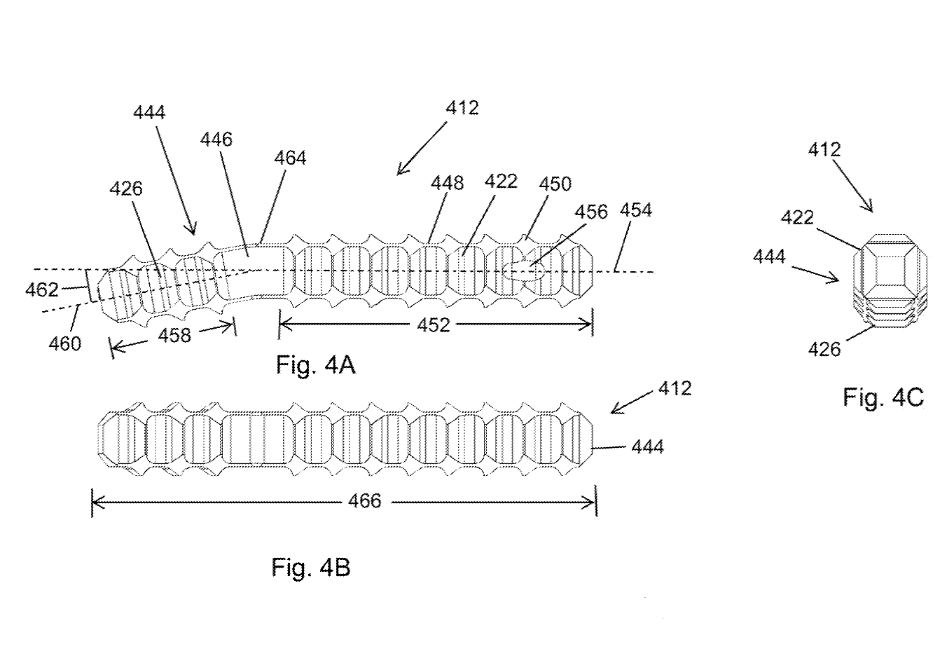

FIG. 4A is a side view of an embodiment of a fusion implant 412 (an “implant”) having features of the present invention. The design of the implant 412 can be varied. In the embodiment illustrated in FIG. 4A, the implant 412 includes an implant body 444 having a first (or proximal) section 422, a second (or distal) section 426, and an intermediate section 446. Alternatively, the implant body 444 can be designed without the intermediate section 446.

It should be noted that the use of the terms “first section” and “second section” is merely for ease of description, and is not intended to limit the scope or breadth of the present invention in any manner.

The first section 422 is adapted to fit within a first receiving aperture, e.g., the first receiving aperture 20 illustrated in FIG. 1, that is created within a first bone part, e.g., the first bone part 14A illustrated in FIG. 1.

In this embodiment, the first section 422 includes a plurality of substantially flat regions 448 and a plurality of ridges 450, such that the flat regions 448 and the ridges 450 alternate along a length 452 of the first section 422. The ridges 450 are provided to inhibit the first section 422 of the implant body 444 from being pulled out of the first receiving aperture of the first bone part. For example, in one embodiment, one or more of the ridges 450 can extend substantially transversely to a longitudinal axis 454 of the first section 422 of the implant body 444. Alternatively, the first section 422 can be designed without any ridges, and the entire length 452 of the first section 422 can be substantially flat.

Additionally, in certain embodiments, the length 452 of the first section 422 can be between approximately eight and fifteen millimeters. For example, in one specific embodiment, the length 452 of the first section 422 can be approximately ten millimeters. In another specific embodiment, the length 452 of the first section 422 can be approximately thirteen millimeters. Alternatively, the length 452 of the first section 422 can be less than eight millimeters or greater than fifteen millimeters.

Further, as illustrated in FIG. 4A, the first section 422 can include an orientation indicator 456 that indicates that the first section 422 is designed to be inserted into the first bone part. For example, in one embodiment, the orientation indicator 456 can be an oval shaped, recessed area in the first section 422 of the implant body 444. With this design, the first section 422 of the implant body 444 will be properly inserted into the first bone part. More specifically, with this design, the physician inserting the implant body 444 will be able to quickly identify which end of the implant 412 is the first section 422 that is to be inserted into the first (or proximal) bone part. Alternatively, the orientation indicator 456 can have a different design.

The second section 426 is adapted to fit within a second receiving aperture, e.g., the second receiving aperture 24 illustrated in FIG. 1, that is created within a second bone part, e.g., the second bone part 16A illustrated in FIG. 1.

In this embodiment, similar to the first section 422, the second section 426 also includes a plurality of substantially flat regions 448 and a plurality of ridges 450, such that the flat regions 448 and the ridges 450 alternate along a length 458 of the second section 426. In one embodiment, one or more of the ridges 450 can extend substantially transversely to a longitudinal axis 460 of the second section 426 of the implant body 444. Alternatively, the second section 426 can be designed without any ridges, and the entire length 458 of the second section 425 can be substantially flat.

Additionally, in certain embodiments, the length 458 of the second section 426 can be between approximately four and eight millimeters. For example, in one specific embodiment, the length 458 of the second section 426 can be approximately six millimeters. Alternatively, the length 458 of the second section 426 can be less than four millimeters or greater than eight millimeters.

Further, as illustrated in this embodiment, the second section 426 can be angled relative to the first section 422. Stated another way, the longitudinal axis 454 of the first section 422 and the longitudinal axis 460 of the second section 426 can define an orientation angle 462 therebetween. For example, in certain embodiments, the orientation angle 462 between the first section 422 and the second section 426 can be between approximately zero degrees and sixty degrees. In one specific embodiment, the orientation angle 462 between the first section 422 and the second section 426 can be approximately ten degrees. Alternatively, the orientation angle 462 between the first section 422 and the second section 426 can be greater than sixty degrees, or the second section 426 can be substantially aligned with the first section 422, i.e. the orientation angle 462 is zero degrees.

The intermediate section 446 extends between the first section 422 and the second section 426. Moreover, the intermediate section 446 is designed to extend substantially between the first bone part and the second bone part, e.g., be positioned at the joint (for example, the first joint 18A illustrated in FIG. 1) when the implant 412 is implanted in the body.

In certain embodiments, the intermediate section 422 can comprise and/or include a depth indicator 464 that indicates when the first section 422 of the implant body 444 is properly inserted into the first (or proximal) bone part and/or when the second section 426 of the implant body 444 is properly inserted into the second (or distal) bone part. For example, the depth indicator 464 can include a joint line feature (e.g., half way between a gap in the teeth of the implant) to indicate an insertion depth of the first section 422 into the first bone part and/or to indicate an insertion depth of the second section 426 into the second bone part. Alternatively, the depth indicator 464 can be at the end of the ridges 450 on the first section 422 of the implant body 444 and/or at the end of the ridges 450 on the second section 426.

With this design, in certain embodiments, the first section 422 of the implant body 444 is inserted by the physician until the depth indicator 464 is approximately flush with the end of the first bone part. This will ensure that the first section 422 is properly inserted to the correct depth. This is particularly important for angled implants to ensure that the angled implant is properly positioned relative to the bone parts. Somewhat similarly, in certain embodiments, the second section 426 of the implant body 444 is inserted by the physician until the depth indicator 464 is approximately flush with the end of the second bone part. This will ensure that the second section 426 is properly inserted to the correct depth.

In some embodiments, one or more of the first section 422, the second section 426 and the intermediate section 446 of the implant body 444 can be made of bone. For example, in certain embodiments, each section 422, 426, 446 of the implant body 444 can be made of cortical bone. Moreover, each section 422, 426, 446 can be made of bone that is partially demineralized. In one embodiment, one or more of the sections 422, 426, 446 can be a partially demineralized human cortical bone allograft.

In certain embodiments, because cortical bone is anisotropic, the cortical bone's longitudinal axis would be aligned with the longitudinal axes 454, 460 of the implant body 444. In some cases it may be optimal to orient the cortical bone in the opposite direction (90°) to take advantage of the mechanical properties of the bone. Other sources of the bone material may be used and include, but is not limited to, bovine.

As provided herein, the implant 412 can have the high strength of cortical bone to support the fusion. It can further have the osteoconductive properties of allograft bone but the surface demineralization will give it osteoinductive properties to help with the fusion process. The demineralization will also make the outside soft so that the implant 412 can be placed into the prepared bone to be treated. The demineralized layer extends from the surface of the bone toward the center of the implant body 444. The demineralized section may extend from the surface to the core depending on the application. In an alternative embodiment, there will not be any demineralization of the outer surface of the implant body 444. This will result in a naturally hard implant 412.

FIG. 4B is a bottom view of the implant 412 illustrated in FIG. 4A. In particular, FIG. 4B illustrates that the implant body 444 has an overall length 466, which can be between approximately twelve and twenty-three millimeters. For example, in one specific embodiment, the overall length 466 of the implant body 444 can be approximately sixteen millimeters. In another specific embodiment, the overall length 466 of the implant body 444 can be approximately nineteen millimeters. Alternatively, the overall length 466 of the implant body 466 can be less than twelve millimeters or greater than twenty-three millimeters.

FIG. 4C is an end view of the implant 412 illustrated in FIG. 4A. As illustrated in FIG. 4C, the implant body 444, i.e. one or more of the first section 422, the second section 426 and the intermediate section 446 (illustrated in FIG. 4A), can have a cross-section that is substantially octagon-shaped. Alternatively, the implant body 444 can have a cross-section that is substantially triangle-shaped, square-shaped, rectangle-shaped, hexagon-shaped, or some other shape. As noted above, with this design, the non-circular cross-sectional shape of the implant body 444 functions to inhibit relative movement between the first section 422 of the implant body 444 and the first receiving aperture of the first bone part, and the second section 426 of the implant body 444 and the second receiving aperture of the second bone part.

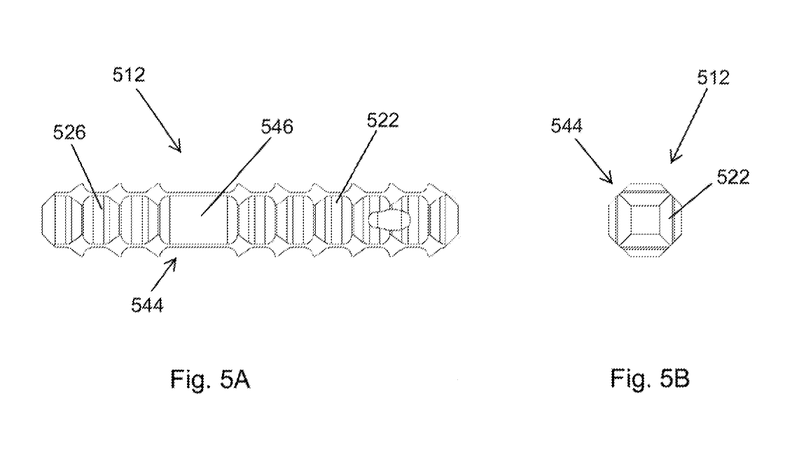

FIG. 5A is a side view of another embodiment of a fusion implant 512 (an “implant”) having features of the present invention. The implant 512 is substantially similar to the implant 412 illustrated and described above in relation to FIGS. 4A-4C. For example, the implant 512 includes an implant body 544 having a first section 522, a second section 526, and an intermediate section 546 that are substantially similar to the first section 422, the second section 426, and the intermediate section 446 illustrated and described above in relation to FIGS. 4A-4C. However, in the embodiment illustrated in FIG. 5A, the first section 522 and the second section 526 of the implant body 544 are substantially aligned. Stated another way, in this embodiment, the implant body 544 is generally straight beam shaped, with no orientation angle (i.e. an orientation angle of zero degrees) between the first section 522 and the second section 526.

FIG. 5B is an end view of the implant 512 illustrated in FIG. 5A. Similar to the previous embodiment, as illustrated in FIG. 5B, the implant body 544, i.e. one or more of the first section 522, the second section 526 (illustrated in FIG. 5A) and the intermediate section 546 (illustrated in FIG. 5A), can have a cross-section that is substantially octagon-shaped. Alternatively, the implant body 544 can have a cross-section that is substantially triangle-shaped, square-shaped, rectangle-shaped, hexagon-shaped, or some other shape. With this design, the non-circular cross-sectional shape of the implant body 544 again functions to inhibit relative movement between the first section 522 of the implant body 544 and the first receiving aperture of the first bone part, and the second section 526 of the implant body 544 and the second receiving aperture of the second bone part.

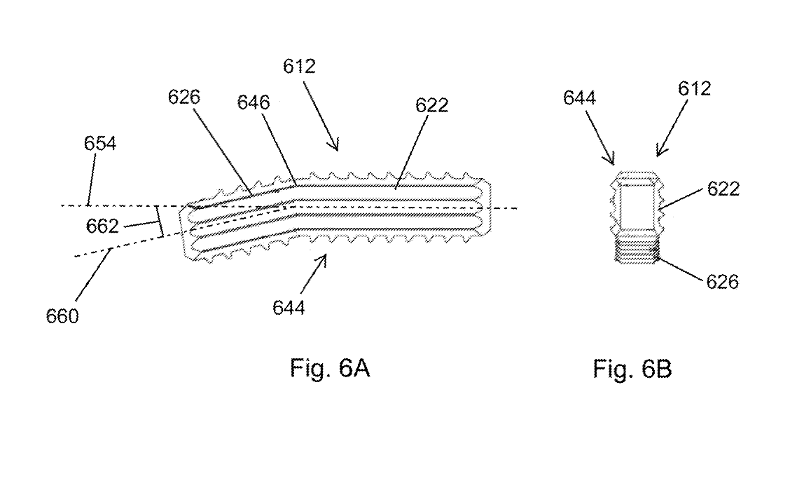

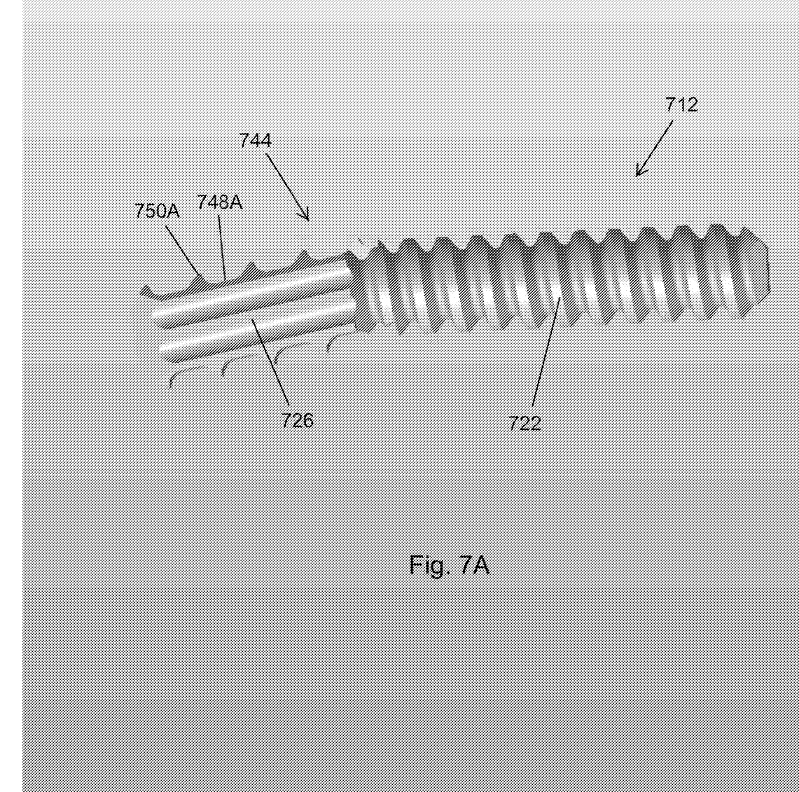

FIG. 6A is a side view of still another embodiment of a fusion implant 612 (an “implant”) having features of the present invention. The implant 612 is somewhat similar to the implants 412, 512 illustrated and described above. For example, the implant 612 includes an implant body 644 having a first section 622, a second section 626, and an intermediate section 646 that are somewhat similar to the first sections 422, 522, the second sections 426, 526, and the intermediate sections 446, 546 illustrated and described above.

As illustrated in FIG. 6A, similar to the embodiment illustrated in FIGS. 4A-4C, the second section 626 is again angled relative to the first section 622. Stated another way, a longitudinal axis 654 of the first section 622 and a longitudinal axis 660 of the second section 626 can define an orientation angle 662 therebetween. For example, in certain embodiments, the orientation angle 662 between the first section 622 and the second section 626 can be between approximately zero degrees and sixty degrees. In one specific embodiment, the orientation angle 662 between the first section 622 and the second section 626 can be approximately ten degrees. Alternatively, the orientation angle 662 between the first section 622 and the second section 626 can be greater than sixty degrees, or the second section 626 can be substantially aligned with the first section 622, i.e. the orientation angle 662 is zero degrees.

FIG. 6B is an end view of the implant 612 illustrated in FIG. 6A. In particular, FIG. 6B illustrates that the implant body 644, i.e. one or more of the first section 622, the second section 626 and the intermediate section 646 (illustrated in FIG. 6A), has a cross-section that is substantially rectangle-shaped. Alternatively, the implant body 644 can have a cross-section that is substantially triangle-shaped, square-shaped, hexagon-shaped, octagon-shaped, or some other shape. With this design, the non-circular cross-sectional shape of the implant body 644 again functions to inhibit relative movement between the first section 622 of the implant body 644 and the first receiving aperture of the first bone part, and the second section 626 of the implant body 644 and the second receiving aperture of the second bone part.

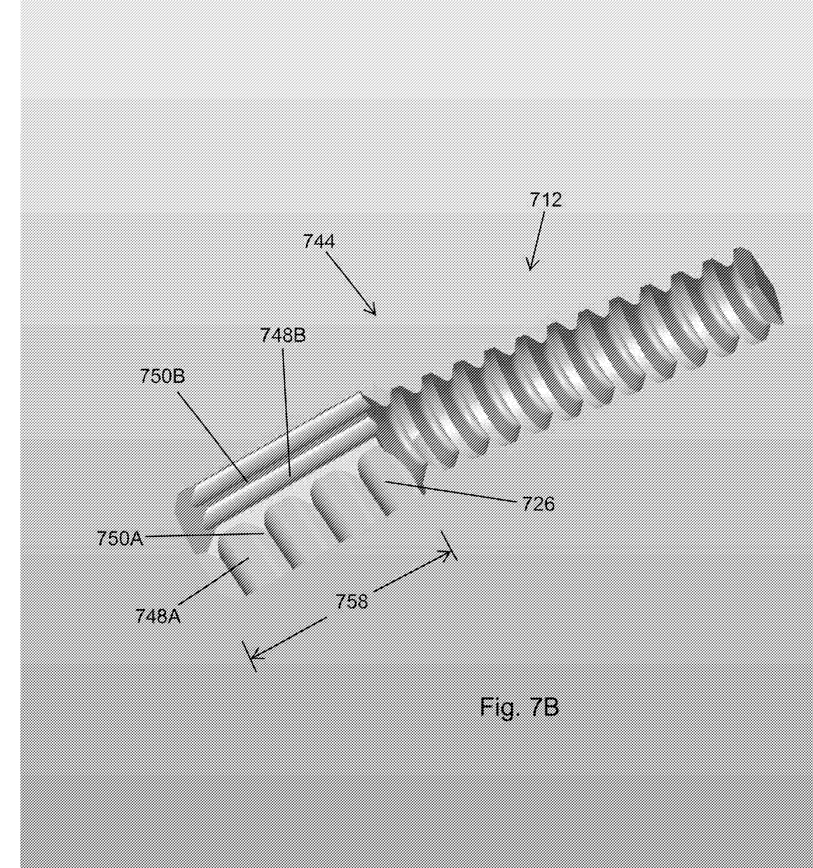

FIG. 7A is a side view of yet another embodiment of a fusion implant 712 (an “implant”) having features of the present invention. The implant 712 is somewhat similar to the implants 412, 512, 612 illustrated and described above. For example, the implant 712 includes an implant body 744 having a first section 722 and a second section 726 that are somewhat similar to the first sections 422, 522, 622 and the second sections 426, 526, 626 illustrated and described above. Additionally, as illustrated in FIG. 7A, similar to the embodiments illustrated in FIGS. 4A-4C and 6A-6B, the second section 726 is again angled relative to the first section 722.

However, in the embodiment illustrated in FIG. 7A, the first section 722 is threaded and includes a substantially circular cross-section to enable the first section 722 to be threaded into a first receiving aperture, e.g., the first receiving aperture 20 illustrated in FIG. 1, that is created within a first bone part, e.g., the first bone part 14A illustrated in FIG. 1.

FIG. 7B is a perspective view of the implant 712 illustrated in FIG. 7A. In particular, FIG. 7B illustrates that the second section 726 of the implant body 744 has a cross-section that is substantially square-shaped or rectangle-shaped. Alternatively, the second section 726 can have a cross-section that is substantially triangle-shaped, hexagon-shaped, octagon-shaped, or some other shape. With this design, the non-circular cross-sectional shape of the second section 726 functions to inhibit relative movement between the second section 726 of the implant body 744 and the second receiving aperture of the second bone part. Still alternatively, the second section 726 can be threaded and/or can include a substantially circular cross-section to enable the second section 726 to be threaded into a second receiving aperture, e.g., the second receiving aperture 24 illustrated in FIG. 1, that is created within a second bone part, e.g., the second bone part 16A illustrated in FIG. 1.

Additionally, FIG. 7B illustrates that the second section 726 of the implant body 744 includes a plurality of first flat regions 748A and a plurality of first ridges 750A, such that the first flat regions 748A and the first ridges 750A alternate along a length 758 of the second section 726. Further, as more clearly illustrated in FIG. 7A, the plurality of first flat regions 748A and the plurality of first ridges 750A can be positioned on each of two opposite sides of the second section 726 of the implant body 744. Alternatively, the second section 726 can include alternating first flat regions 748A and first ridges 750A on more than two sides or less than two sides of the second section 726.

Moreover, FIG. 7B illustrates that the second section 726 includes a plurality of second flat regions 748B and a plurality of second ridges 750B that alternate such that each of the plurality of second flat regions 748B and the plurality of second ridges 750B extend in a direction substantially parallel to the length 758 of the second section 726. In one embodiment, the plurality of second flat regions 748B and the plurality of second ridges 750B can be positioned on each of two opposite sides of the second section 726 of the implant body 744. Alternatively, the second section 726 can include alternating second flat regions 748B and second ridges 750B on more than two sides or less than two sides of the second section 726.

While a number of exemplary aspects and embodiments of a fusion implant 412 have been shown and disclosed herein above, those of skill in the art will recognize certain modifications, permutations, additions and sub-combinations thereof. It is therefore intended that the fusion implant 412 shall be interpreted to include all such modifications, permutations, additions and sub-combinations as are within their true spirit and scope, and no limitations are intended to the details of construction or design herein shown.

1. An implant that facilitates the fusion of a first bone part with a second bone part, the first bone part including a first receiving aperture and the second bone part including a second receiving aperture, the implant comprising:
an implant body that extends between the first bone part and the second bone part, the implant body including a first section that fits within the first receiving aperture in the first bone part, and a second section that fits within the second receiving aperture in the second bone part, wherein at least one of the first section and the second section is made of bone, and wherein at least one of the first section and the second section is formed to have a non-circular cross-sectional shape to inhibit relative motion between the bone parts and the implant body.

2. The implant of claim 1 wherein each of the first section and the second section are made of bone.

3. The implant of claim 1 wherein each of the first section and the second section are formed to have a non-circular cross-sectional shape to inhibit relative motion between the bone parts and the implant body.

4. The implant of claim 1 wherein one of the first section and the second section is threaded and includes a substantially circular cross-section that is threaded into its corresponding receiving aperture.

5. The implant of claim 1 wherein the implant body includes a plurality of ridges, and wherein at least one of the ridges extends substantially transverse to a longitudinal axis of the implant body.

6. The implant of claim 1 wherein at least one of the first section and the second section has a generally rectangular cross-sectional shape.

7. The implant of claim 1 wherein at least one of the first section and the second section has a generally octagonal cross-sectional shape.

8. The implant of claim 1 wherein at least one of the first section and the second section is made of cortical bone.

9. The implant of claim 1 wherein at least one of the first section and the second section is made of bone that is partially demineralized.

10. The implant of claim 1 wherein the implant body is generally straight beam shaped.

11. The implant of claim 1 wherein the second section is angled relative to the first section.

12. The implant of claim 1 wherein the implant body includes an orientation indicator that indicates that the first section of the implant body is to be inserted into the first bone part.

13. The implant of claim 1 wherein the implant body includes a depth indicator that indicates when the first section is properly inserted into the first bone part.

14. The implant of claim 13 wherein the depth indicator includes a joint line feature to indicate an insertion depth of the first section into the first bone part.

15. An implant that facilitates the fusion of a first bone part with a second bone part, the first bone part including a first receiving aperture and the second bone part including a second receiving aperture, the implant comprising:
an implant body that extends between the first bone part and the second bone part, the implant body including a first section that fits within the first receiving aperture in the first bone part, and a second section that fits within the second receiving aperture in the second bone part, wherein at least one of the first section and the second section is made of bone, and wherein at least one of the first section and the second section is threaded and includes a substantially circular cross-section that is threaded into its corresponding receiving aperture.

16. The implant of claim 15 wherein each of the first section and the second section is threaded and includes a substantially circular cross-section, such that the first section is threaded into the first receiving aperture and the second section is threaded into the second receiving aperture.

17. The implant of claim 15 wherein at least one of the first section and the second section is made of cortical bone.

18. (canceled)

19. (canceled)

20. (canceled)
